(19) United States
(12) Patent Application Publication
(10) Pub. No.: US 2026/0256599 A1
(43) Pub. Date: Sep. 3, 2026

(54) SUPPORTS WITH CONNECTION MEMBERS FOR ANTENNAS OF BODILY IMPLANTS

(71) Applicant: Boston Scientific Scimed, Inc., Maple Grove, MN (US)

(72) Inventors: Hannah Mitchell, Minneapolis, MN (US); Matthew William Couri, Plymouth, MN (US); Trey Henry Achterhoff, Arden Hills, MN (US); Derek John Boettger, Woodbury, MN (US)

(21) Appl. No.: 19/549,636

(22) Filed: Feb. 25, 2026

Related U.S. Application Data
(60) Provisional application No. 63/766,126, filed on Mar. 3, 2025.

(51) Int. Cl. A61F 2/48; A61F 2/00; A61F 2/26

(52) U.S. Cl. CPC A61F 2/482; A61F 2/004; A61F 2/26

(57) ABSTRACT

A device includes a housing, an electronic control system, a recharge antenna and a support member. The housing defines a cavity. The housing has an engagement portion. The electronic control system is at least partially disposed within the cavity defined by the housing. The support member has an engagement portion. The engagement portion of the support member is configured to engage the engagement portion of the housing to couple the support member to the housing.

### CROSS-REFERENCE TO RELATED APPLICATION

This application claims priority to U.S. Provisional Patent Application No. 63/766,126, filed on March 3, 2025, entitled “SUPPORTS WITH CONNECTION MEMBERS FOR ANTENNAS OF BODILY IMPLANTS”, the disclosure of which is incorporated by reference herein in its entirety.

### TECHNICAL FIELD

This disclosure relates generally to bodily implants, and more specifically to bodily implants that include supports or support members for antennas.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. For example, flow of fluid may be directed to an inflatable member to place the inflatable member in an inflated configuration. Similarly, flow of fluid may be directed away from the inflatable member to place the inflatable member in a deflated configuration. In some implantable fluid-operated devices, an implantable pumping device may be electrically operated. For example, in some devices, the implantable pumping device may be electrically operated using a device located outside of the body of the patient. Additionally, some such devices include antennas that allow for charging of the bodily implant and/or communication or control of the bodily implant from a device located outside of the body of the patient.

There is a need for bodily implant that efficiently supports a charging antenna.

### SUMMARY

According to a general aspect, a device includes a housing, an electronic control system, a recharge antenna and a support member. The housing defines a cavity. The housing has an engagement portion. The electronic control system is at least partially disposed within the cavity defined by the housing. The support member has an engagement portion. The engagement portion of the support member is configured to engage the engagement portion of the housing to couple the support member to the housing.

In some implementations, the support member defines a groove, the groove is configured to receive at least a portion of the recharge antenna.

In some implementations, the bodily implant includes a fluid control system, at least a portion of the fluid control system is disposed within the cavity defined by the housing. In some implementations, the bodily implant includes an inflatable member configured to be placed in an inflated configuration and a deflated configuration. In some implementations, the bodily implant includes a fluid reservoir and an inflatable member.

In some implementations, the bodily implant includes a fluid reservoir operatively coupled to the housing, and the bodily implant includes an inflatable member operatively coupled to the housing.

In some implementations, the bodily implant is a penile implant. In some implementations, the bodily implant is an artificial sphincter.

In some implementations, the coupling portion of the support member includes a projection, the projection has a stem portion and a head portion. In some implementations, the coupling portion of the support member includes a projection, the projection has a stem portion and a head portion, the stem portion of the projection has a first size, the head portion of the projection has a second size, the second size being larger than the first size.

In some implementations, the coupling portion of the housing includes a slot, the slot is configured to receive the coupling portion of the support member. In some implementations, the coupling portion of the housing includes a slot, the slot includes a first portion having a first size and a second portion having a second size, the first size being larger than the second size.

In some implementations, the coupling portion of the housing is configured to receive a portion of the coupling portion of the support member to couple the support member to the housing.

In some implementations, the coupling portion of the support member is configured to receive a portion of the coupling portion of the housing to couple the support member to the housing.

In some implementations, the coupling portion of the housing includes a projection, the coupling portion of the support member includes an opening, the opening being configured to receive a portion of the projection of the coupling portion of the housing.

According to another aspect, a bodily implant includes a housing defining a cavity, the housing defining a slot; an electronic control system at least partially disposed within the cavity; a recharge antenna; and a support member having a projection, the slot being configured to receive at least a portion of the projection to couple the support member to the housing.

In some implementations, the projection of the support member has a stem portion and a head portion, the stem portion of the projection has a first size, the head portion of the projection has a second size, the second size being larger than the first size. In some implementations, the slot includes a first portion having a first size and a second portion having a second size, the first size being larger than the second size.

In some implementations, the support member defines a groove, the groove being configured to receive at least a portion of the recharge antenna.

In some implementations, the bodily implant includes a fluid reservoir and an inflatable member.

### BRIEF DESCRIPTION OF THE DRAWINGS

FIG. 1 is a block diagram of an implantable fluid-operated inflatable device.

FIG. 2A illustrates a system including an example implantable fluid-operated inflatable device.

FIG. 2B illustrates a system including an example implantable fluid-operated inflatable device.

FIG. 3 is a schematic diagram of a fluidic architecture of an implantable fluid-operated inflatable device.

FIG. 4A is an exploded view of a portion of an example implantable fluid-operated inflatable device.

FIG. 4B is a perspective view of a portion of a housing of the implantable fluid-operated inflatable device of FIG. 4A.

FIG. 5A is an exploded view of a portion of the implantable fluid-operated inflatable device of FIG. 4A.

FIG. 5B is a perspective view of a portion of the support of FIG. 4A

FIG. 6 is an exploded view of a portion of the implantable fluid-operated inflatable device of FIG. 4A.

FIG. 7 is a side view of a support member according to an implementation.

FIGS. 8-9 are perspective views of a portion of a housing according to an implementation.

FIGS. 10-12 are side views of the support member of FIG. 7 and the housing of FIGS. 8-9.

FIG. 13 is a cross-sectional view of a portion of the support member of FIG. 7 and a portion of the housing of FIGS. 8-9.

FIG. 14 is a perspective view of a support member according to an implementation and a portion of a housing according to an implementation.

FIG. 15 is a perspective view of a portion of a housing according to an implementation.

FIG. 16 is a perspective view of a support member according to an implementation.

FIG. 17 is a perspective view of a portion of the housing of FIG. 15 and a portion of the support member of FIG. 16.

FIG. 18 is a perspective view of a portion of a housing according to an implementation.

FIG. 19 is a perspective view of a portion of a support member.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure. The term physician may hereinafter be used for a person who places or implants a device within the body of the patient or otherwise provides instructions or counsel to the patient on how to use the device disposed within the body of the patient.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically operated.

For example, the pumps and/or valves of the fluid control system can be electronically operated by the fluid control system to control the pressure of, and the flow of fluid in, parts of the fluid-operated inflatable device. An electronically-operated fluid control system, in accordance with implementations described herein, can include a plurality of electromechanical devices, such as, piezoelectric devices that operate as pumps or as valves in the system. One or more processors or controllers can control the electromechanical devices.

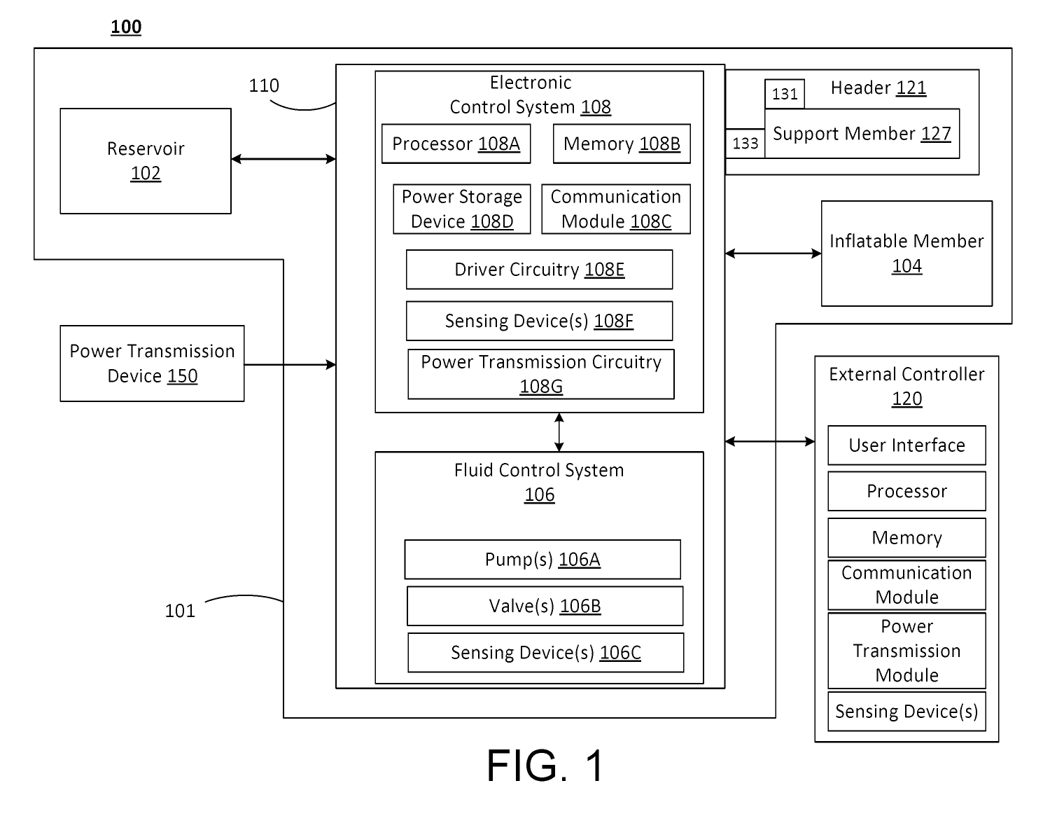

FIG. 1 is a block diagram of an example implantable fluid-operated inflatable device 100. The device 100 includes components that are configured to be implanted or disposed within a body of a patient or user and components that are configured to operate and be disposed outside of the body of the patient or user. The implantable portion 101 (the portion that is configured to be disposed within the body of the patient) includes a reservoir 102, an electronic control system 108, a fluid control system 106, and an inflatable member 104. The portions that are configured to be disposed outside of the body of the patient include a power transmission device 150 and an external controller 120.

In the example inflatable device 100, the electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104. The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, a power storage device 108D (e.g., a battery), electronic driver circuity 108E, sensing devices 108F, such as, for example, voltage measurement circuitry, current measurement circuitry, an accelerometer, and other such components configured to provide for the monitoring, operation, and control of the implantable fluid-operated inflatable device 100, and power transmission circuitry 108G. In some examples, the communication module 108C of the electronic control system 108 may provide for communication with one or more external devices such as, for example, the external controller 120.

In some examples, the external controller 120 includes components such as, for example, a user interface, a processor, a memory, a communications module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like. In some implementations, the communications modules are configured to communicate via Bluetooth. In other implementations, the communication modules are configured to communicate via a different method such as via Wi-Fi.

In some implementations, the power transmission module 108G may include a charging antenna configured to receive power from an external device. Additionally, in some implementations, the communication module 108C may include a communications antenna. The charging antenna and the communications antenna may be disposed within the body. In the illustrated implementation, the device 100 includes a header portion 121 that extends from housing 110. In some implementations, the header portion 121 houses at least a portion of the charging antenna 123 and at least a portion of the communications antenna 125. In the illustrated implementation, the device 100 also includes a support member 127. The support member 127 is disposed within the header portion 121 and is configured to engage the charging antenna 123. In some implementations, the support member 127 provides support to the charging antenna 123, for example during the manufacturing process. Additionally, in some implementations, the support member 127 is configured to keep a first portion of the charging antenna 123 spaced from a second portion of the charging antenna 123. Additionally, in some implementations, the support member 127 may also engage the communications antenna 125. In some implementations, the support member 127 is coupled to the housing 110. For example, the support member 127 may be coupled to a top surface of the housing 110. In some implementations, the support member 127 includes an engagement portion 131 that is configured to engage an engagement portion 133 of the housing 110 to couple the support member 127 to the housing 110. In some implementations, the engagement portion 131 of the support member 127 includes a projection. In other implementations, the engagement portion 131 of the support member 127 includes another structure such as an opening or cavity. In some implementations, the engagement portion 133 of the housing 110 includes an opening or a slot. In other implementations, the engagement portion 133 of the housing 110 includes another structure such as a projection.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing 110. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

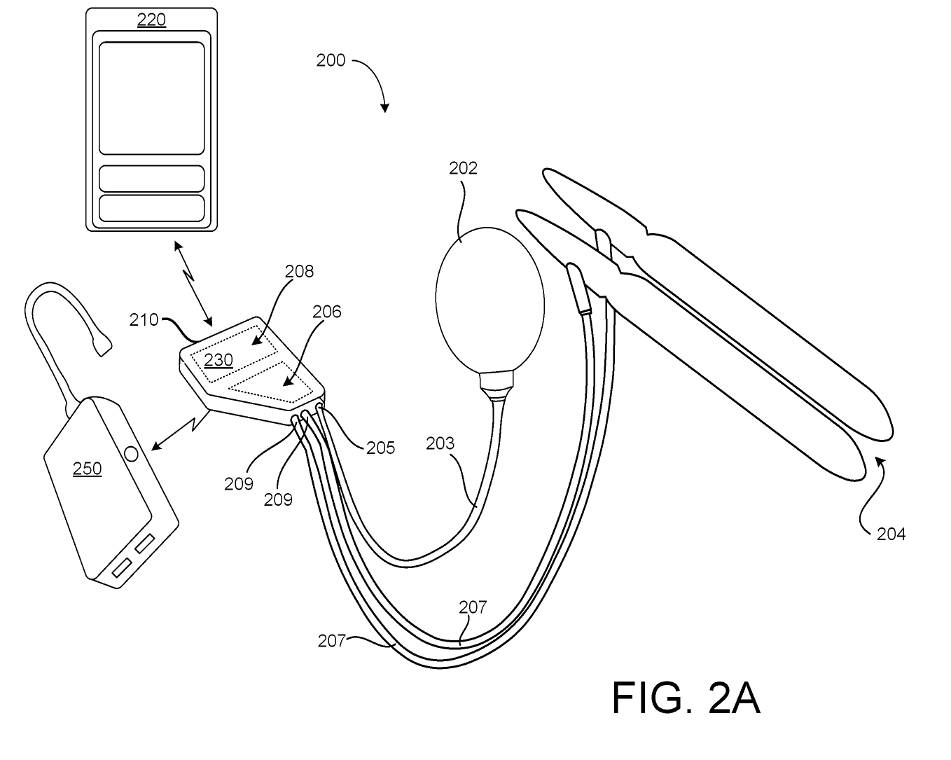

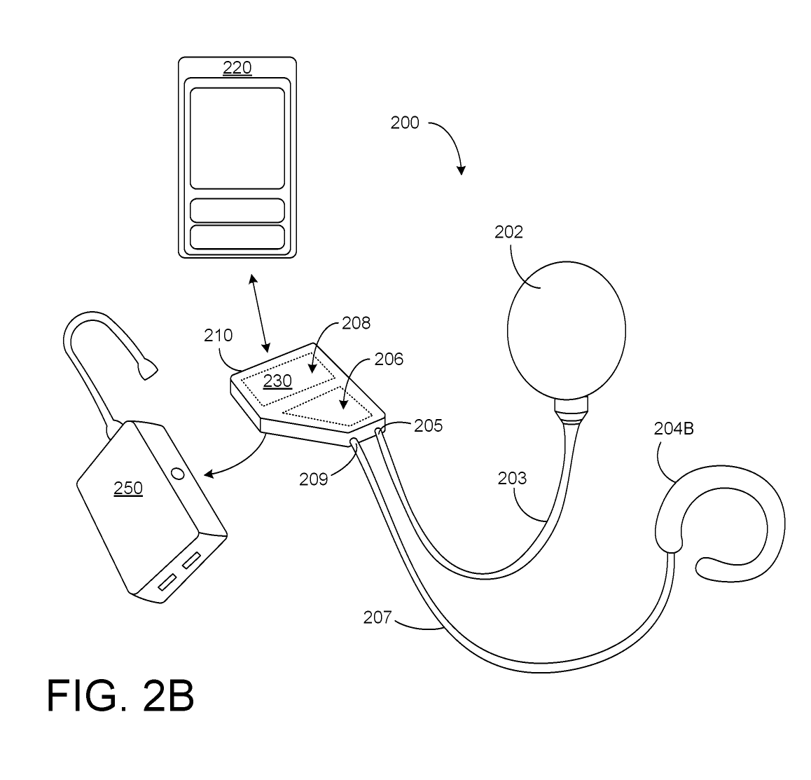

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2A. In another example, the implantable fluid-operated inflatable device 100 as shown in FIG. 2B may be representative of an artificial sphincter (such as an artificial urinary sphincter) that includes an inflatable member 204B that forms a loop or cuff. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like. In other implementations, the implantable device may be an electrostimulation device, neuromodulation device, or another other type of implantable device.

An example system including an example implantable fluid-operated inflatable device 200 in the form of an example inflatable penile prosthesis is shown in FIG. 2A. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2A, the inflatable member 204 is in the form of a pair of inflatable cylinders. In the example shown in FIG. 2A, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the examples shown in FIG. 2A and FIG. 2B, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules.

In some implementations, an application stored in a memory and executed by a processor of the external controller 220 may allow the patient or user to operate, view, monitor and alter operation of the inflatable device 200. For example, the patient or user may be able to use the external controller 220 to cause the inflatable device 200 (or the inflatable member) to be placed in its inflated configuration or may be able to cause the inflatable device 200 (or the inflatable member) to be placed in its deflated configuration.

In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid-operated inflatable device, in the form of the example inflatable penile prostheses shown in FIG. 2A, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control and physician control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and one or more valve devices and one or more sensing devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

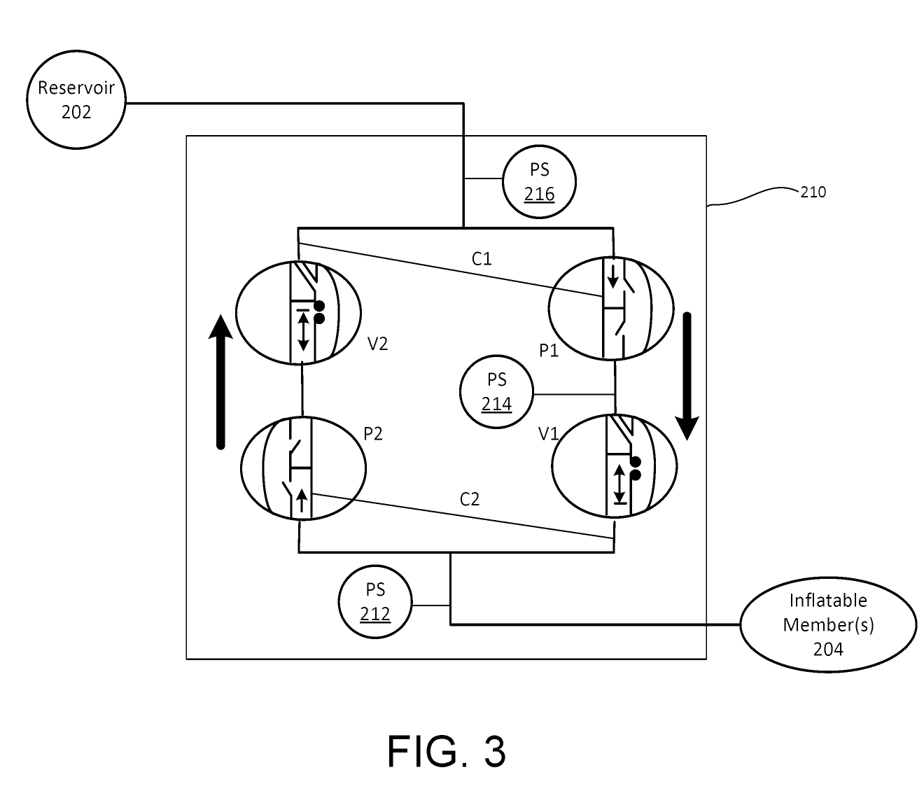

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In an example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material out from of the section of the first fluid passageway when fluid is pumped from the inflatable member 204 to the reservoir 202. In an example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material out from of the section of the second fluid passageway when fluid is pumped from the reservoir 202 to the inflatable member 204.

In some implementations, the example fluidic architecture can include one or more pressure sensors 212, 214, 216, each configured to measure a fluid pressure at a point in the system. For example, a first pressure sensor 212 can be connected to a fluidic passageway, conduit, chamber or component located fluidically between the inflatable member 204 and pumps P1, P2 and valves V1, V2, and can be configured to measure a fluid pressure at this location, which can also serve as a measure of a fluid pressure in the inflatable member(s) 204, because the fluid is essentially incompressible and the conduit between the pressure sensor 212 and the inflatable member(s) 204 can be considered to be free of obstruction. A second pressure sensor 214 can be connected to a fluidic passageway, conduit, chamber or component located fluidically between pump P1 and valve V1 and can be configured to measure a fluid pressure at this location. A third pressure sensor 216 can be connected to a fluidic passageway, conduit, chamber or component located fluidically between the reservoir 202 and pumps P1, P2 and valves V1, V2, and can be configured to measure a fluid pressure at this location, which can also serve as a measure of a fluid pressure in the reservoir, because the fluid is essentially incompressible and the conduit between the pressure sensor 216 and the reservoir 202 can be considered to be free of obstruction. In some implementations one or more of the pressure sensors 212, 214, 216 can be contained with the housing 210.

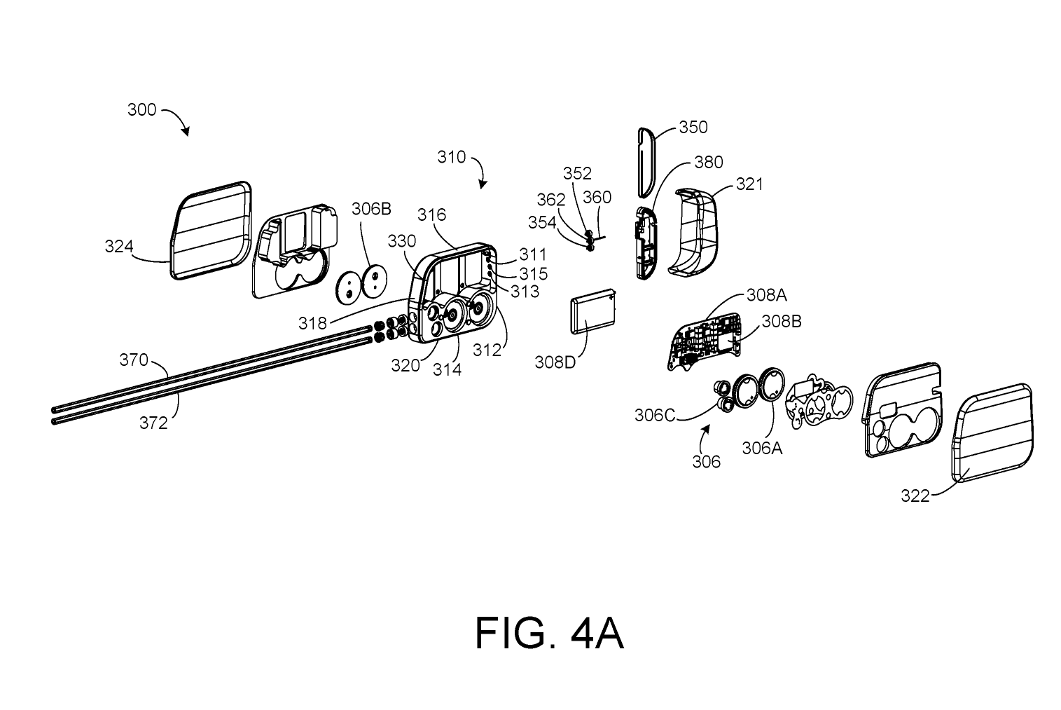

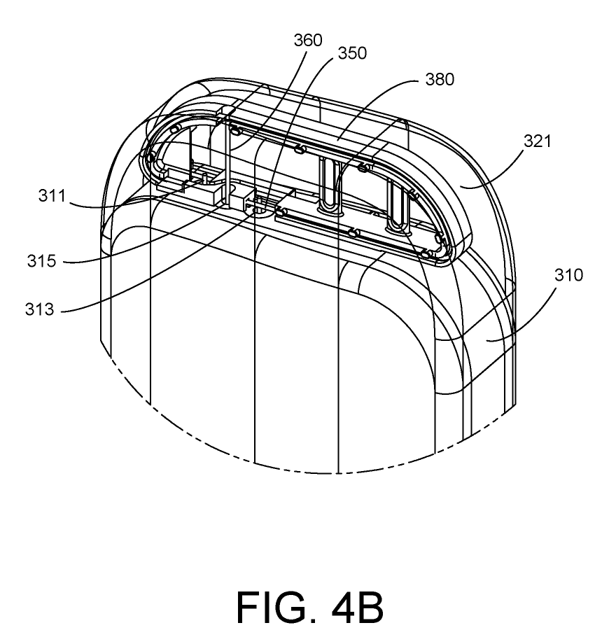

FIG. 4A is an exploded view of a portion of an example implantable fluid-operated inflatable device 300. FIG. 4B is a perspective view of a portion of a housing 310 of the implantable fluid-operated inflatable device 300. The housing 310 includes a top portion 312, a first side portion 314, a second side portion 316, and a bottom portion 318. The top portion 312, the first side portion 314, the second side portion 316, and the bottom portion 318 collectively define a cavity 330. In the illustrated implementation, the housing 310 also includes a first edge member or front portion 322 and a second edge member or rear portion 324. The first edge member or front portion 322 and the second edge member or rear portion 324 also along with the top portion 312, the first side portion 314, the second side portion 316, and the bottom portion 318 define the cavity 330.

In the illustrated implementation, the housing 310 is formed of three individual pieces or members. Specifically, the housing 310 is formed of the center member 320, the first edge member 322, and the second edge member 324. The center member 320 includes the top portion 312, the first side portion 314, the second side portion 316, and the bottom portion 318. In the illustrated implementation, the center member 320 is unitarily formed (or is monolithic or molded or formed of a single piece of material). In the illustrated implementation, the first edge member 322 and the second edge member 324 are also each unitarily formed. In other implementations, any or all of the center member 320, the first edge member 322, and the second edge member 324 may be formed of more than one piece or members. In some implementations, the center member 320, the first edge member 322, and the second edge member 324 are formed of a metal material. For example, in some implementations, the center member 320, the first edge member 322, and the second edge member 324 are formed of titanium. In other implementations, such members are formed of a different type of metal material. In yet other implementations, such members are formed of a different material.

In some implementations, the center member, the first edge member, and the second edge member are coupled and hermetically sealed.

In the illustrated implementation, the housing 310 has a specific shape. In other implementations, the housing 310 has a shape other than as illustrated.

In the illustrated implementation, the cavity 330 of the housing 310 houses or contains at least a portion of an electronic system 308 of the device 300. For example, in the illustrated implementation, the processor 308A, the memory 308B, and the power storage device 308D are disposed within the cavity 330. In other implementations, other components of an electrical system are disposed within or at least partially disposed within the cavity 330.

In the illustrated implementation, the cavity 330 of the housing 310 houses or contains at least a portion of a fluid control system 306 of the device 300. For example, in the illustrated implementation, the pumps 306B, the valves 306A, and the sensing devices 306C are disposed within the cavity 330. In other implementations, other components of a fluid control system are disposed within or at least partially disposed within the cavity 330.

In the illustrated implementation, the housing 310 is operatively coupled to a reservoir (such as reservoir 102 or 202). Specifically, housing 310 is coupled to the reservoir via a tubular member 370. Similarly, the housing 310 is also operatively coupled to an inflatable member (such as inflatable member 104 or 204). The housing 310 is coupled to the inflatable member via a tubular member 372.

In the illustrated implementation, the device 300 includes a first antenna 350 and a second antenna 360. The first antenna or charging antenna 350 may be a recharge antenna that is configured to interact with an external power transmission device such as power transmission device 150. In some implementations, the power storage device 308D may be recharged via the charging antenna 350. The second antenna or communications antenna 360 may be a communications antenna that is configured to interact with an external control device such as external controller 120. In some implementations, the device 300 may be controlled from outside of a body of a patient via the communications antenna and the instructions or communications received by the communications antenna from the external control device.

In the illustrated implementation, the first antenna 350 and the second antenna 360 extend from the housing 310. Specifically, the first antenna 350 includes a first portion 352 that extends through a first opening 311 defined by the top portion 312 of the housing 310. The first antenna 350 also includes a second portion 354 that extends through a second opening 313 defined by the top portion 312 of the housing 310. The portions of the first antenna 350 that extend into the cavity 330 of the housing 310 may be operatively coupled to power transmission circuitry (such as power transmission circuitry 108G) or other circuitry to harvest power received by the first antenna 350 to thereby charge the power storage device 108D.

In the illustrated implementation, the second antenna 360 includes a first portion 362 that extends through a third opening 315 defined by the top portion 312 of the housing 310. The portion of the second antenna 360 that extends into the cavity 330 of the housing 310 may be operatively coupled to a communications module (such as communication module 108C) to provide communications received from outside of the body of the patient to the device 300.

In the illustrated implementation, the portion of the first antenna 350 and the portion of the second antenna 360 that are disposed outside of the cavity 330 of the housing 310 are disposed within a material that forms a header portion 321. For example, the material may be a transparent material such as silicone or a plastic material.

In the illustrated implementation, the device 300 includes a support member 380. The support member 380 is configured to engage the charging antenna 350. In some implementations, the support member 380 is configured to engage the charging antenna 350 to provide support to the charging antenna 350 during the manufacturing process. For example, the support member 380 may provide support to the antenna while the silicone or other transparent material is disposed around the charging antenna 350. Additionally, in some implementations, the support member 380 is configured to keep or help keep a first portion of the charging antenna 350 spaced from a second portion of the charging antenna 350.

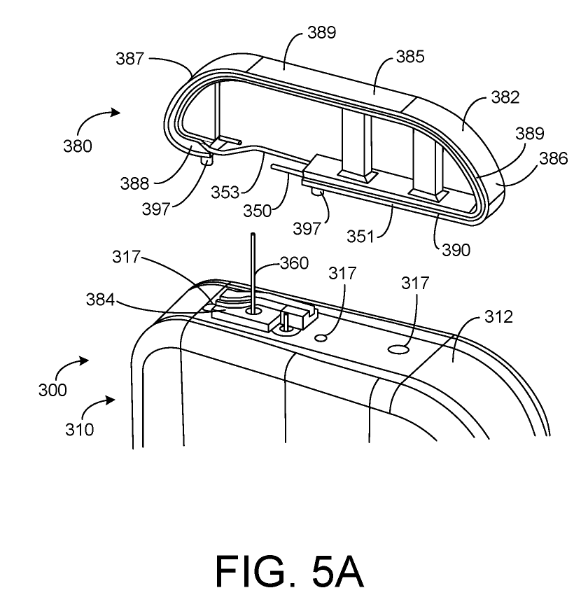

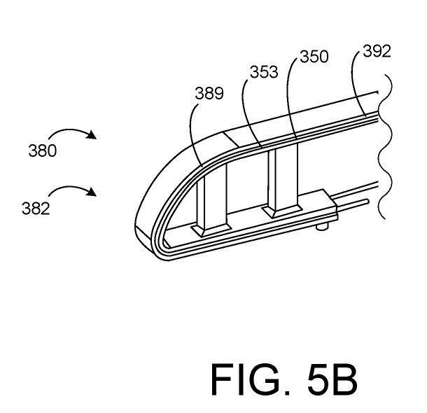

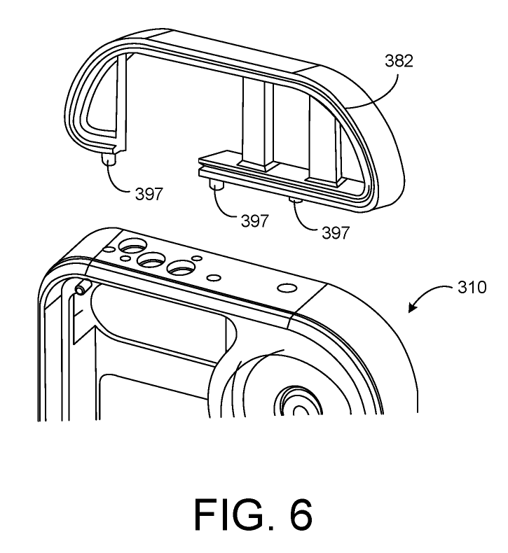

As best illustrated in FIGS. 5A, 5B, and 6, the support member 380 includes a first member 382 and a second member 384. The first member 382 includes a top portion 385, a first end portion 386, a second end portion 387, a first side portion 388, and a second side portion 389. The first end portion 386 is disposed opposite the second end portion 387. Similarly, the first side portion 388 is disposed opposite the second side portion 389.

The first member 382 defines a first groove 390 and a second groove 392. The first groove 390 is disposed on the first side portion 388 of the first member 382. The second groove 392 is disposed on the second side portion 389 of the first member 382.

In the illustrated implementation, a first portion 351 of the charging antenna 350 is disposed within the first groove 390. A second portion 353 of the charging antenna 350 is disposed within the second groove 392. The first portion 351 of the charging antenna 350 thus is disposed apart from or is spaced from the second portion 353 of the charging antenna 350. Specifically, a portion of the first member 382 of the support member 380 is disposed between the first portion 351 of the charging antenna 350 and the second portion 353 of the charging antenna 350.

In some implementations, the first portion 351 of the charging antenna 350 is configured to be coupled within the first groove 390. For example, in some implementations, the first portion 351 of the charging antenna 350 is configured to be frictionally coupled within the first groove 390. In other implementations, another coupling mechanism, such as an adhesive or other material, may be used to help retain the first portion 351 of the charging antenna 350 within the first groove 390.

As best illustrated in FIG. 5A, in the illustrated implementation, the charging antenna 350 is not disposed on the top portion or surface 385. Additionally, the charging antenna 350 is not disposed either of the first end portion or surface 386 or the second end portion or surface 387.

In some implementations, the first groove 390 includes a first sidewall 393, a second sidewall 394, and an end wall 395 disposed between the first sidewall 393 and the second sidewall 394. As discussed above, the first groove 390 is configured to receive a portion of the charging antenna 350. FIG. 5B is a perspective view from a second side of the first portion 382 of the support 380. As best illustrated in FIG. 5B, the second groove 392 is configured similar to the first groove 390.

As best illustrated in FIG. 5A, the second member 384 of the support member 380 defines an opening or through hole 383. The opening or through hole 383 is configured to receive a portion of the communications antenna 360. Specifically, in the illustrated implementation, the communications antenna 360 extends through the opening or through hole 383.

In the illustrated implementation, the support member 380 is coupled to the top surface or portion 312 of the housing 310. Specifically, the first member 382 of the support member 380 includes projections 397 that are configured to be received by cavities or openings 317 of defined by the top surface or portion 312 of the housing 310. In some implementations, the projections 397 are configured to be frictionally coupled within the cavities or openings 317 to couple or help couple the first member 382 of the support member 380 to the housing 310. In other implementations, other coupling mechanisms, such as an adhesive, may be used to couple the first member 382 of the support member 380 to the housing 310.

In some implementations, the second member 384 of the support member 380 is coupled to the top surface or portion 312 of the housing 310. The second member 384 may be coupled to the housing 310 in the same manner as the first member 382 is coupled to the housing 310. For example, the second member 384 may include projections that are configured to be received by cavities or openings defined by the housing 310.

In some implementations, once the charging antenna 350 is coupled to the support member 380 and the support member 380 is coupled to the housing 310, the header portion 321 may be formed around the charging antenna 350, the communication antenna 360, and the support member 380. For example, a material, such as silicone, may be placed or disposed around the charging antenna 350, the communication antenna 360, and the support member 380 to form the header portion 321.

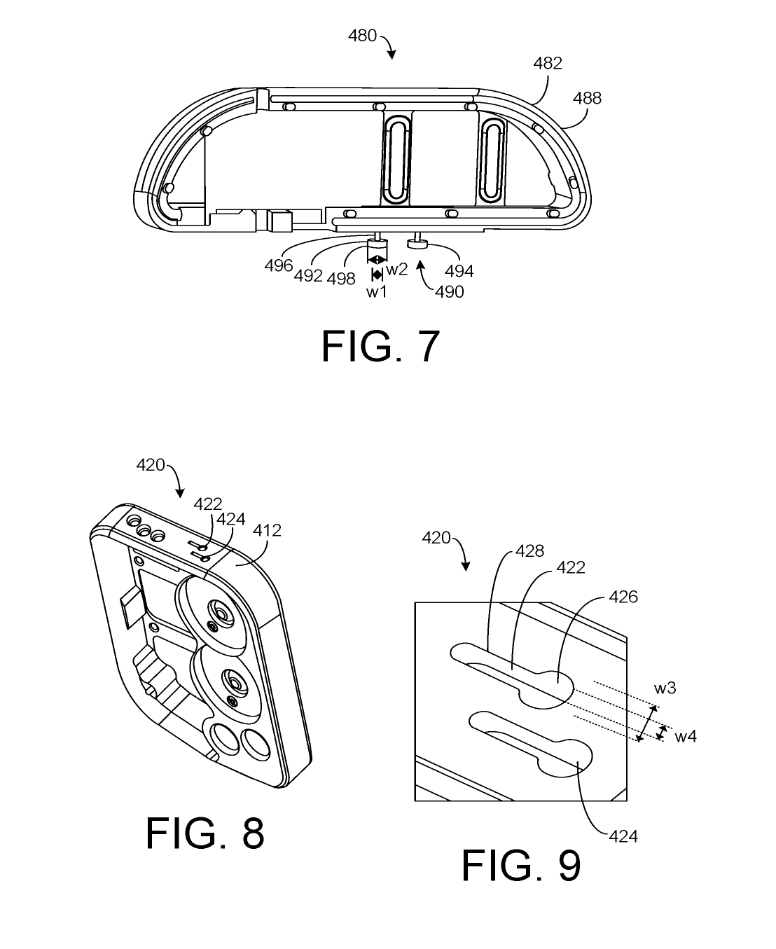

FIG. 7 is a side view of a support member 480. FIGS. 8-9 are perspective views of a portion of a housing 410. As described for the above implementations, the support member 480 is disposed within a header portion and is configured to engage a charging antenna. In some implementations, the support member 480 is configured to engage the charging antenna to provide support to the charging antenna during the manufacturing process. For example, the support member 480 may provide support to the antenna while the silicone or other transparent material is disposed around the charging antenna. Additionally, in some implementations, the support member 480 is configured to keep or help keep a first portion of the charging antenna spaced from a second portion of the charging antenna.

In the illustrated implementation, the support member 480 is unitarily or monolithically formed. In other words, the support member 480 may be formed of a single piece of material. In some implementations, the support member is formed of a non-conductive and/or biocompatible material. For example, the support member 480 may be formed of a plastic material. In other implementations, the support member 480 is formed of more than one piece of material.

The support member 480 defines a first groove 482 and a second groove (not illustrated in FIG. 7). The first groove 482 is disposed on a first side portion 488 of the support member 480. The second groove is disposed on a second side portion (opposite the first side portion) of the support member 480.

In some implementation, a first portion of the charging antenna is disposed within the first groove 482. A second portion of the charging antenna is disposed within the second groove. The first portion of the charging antenna may thus be disposed apart from or spaced from the second portion of the charging antenna. Specifically, a portion of the support member 480 is disposed between the first portion of the charging antenna and the second portion of the charging antenna.

In some implementations, the first portion of the charging antenna is configured to be coupled within the first groove 482. For example, in some implementations, the first portion of the charging antenna is configured to be frictionally coupled within the first groove 482. In other implementations, another coupling mechanism, such as an adhesive or other material, may be used to help retain the first portion of the charging antenna within the first groove 482.

In the illustrated implementation, the support member 480 is coupled to a top surface or portion 412 of the housing 410. Specifically, the support member 480 includes an engagement portion 490 that is configured to engage an engagement portion 420 of the housing 410. For example, the engagement portion 490 of the support member 480 may engage the engagement portion 420 of the support member 480 to couple the support member 480 to the housing 410.

The engagement portion 490 of the support member 480 includes a first projection 492 and a second projection 494. In the illustrated implementation, the first projection 492 and the second projection 494 are functionally and structurally similar. Accordingly, only the first projection 492 will be described in detail but it should be understood that the second projection 494 is similar. The first projection 492 includes a stem portion 496 and a head portion 498. The stem portion 496 is disposed between the head portion 498 and the top surface 412 of the housing 410. The stem portion 496 has a size, such as the width W1 of the stem portion 496, that is smaller than a size, such as the width W2, of the head portion 498.

In some implementations, the first projection 492 and the second projection 494 extend unitarily or monolithically from a body of the support member 480. In some implementations, the first projection 492 and the second projection 494 are made of the same material as the body of the support member.

In the illustrated implementation, the engagement portion 420 of the housing 410 includes a first slot or opening 422 and a second slot or opening 424. In the illustrated implementation, the first slot or opening 422 and the second slot or opening 424 are functionally and structurally similar. Accordingly, only the first slot or opening 422 will be described in detail but it should be understood that the second slot or opening 424 is similar. The slot 422 has a first portion 426 and a second portion 428. The first portion 426 has a size, such as a width W3. The second portion 428 of the slot 422 has a size, such a width W4. The size or width W3 of the first portion 426 slot 422 is larger than the size or width W4 of the second portion 428 of the slot 422.

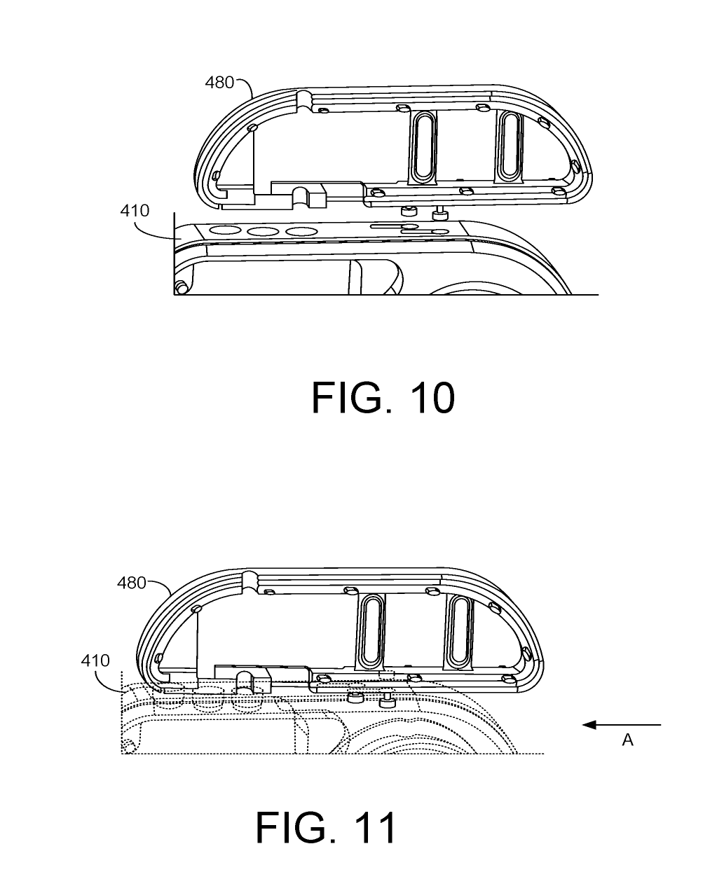

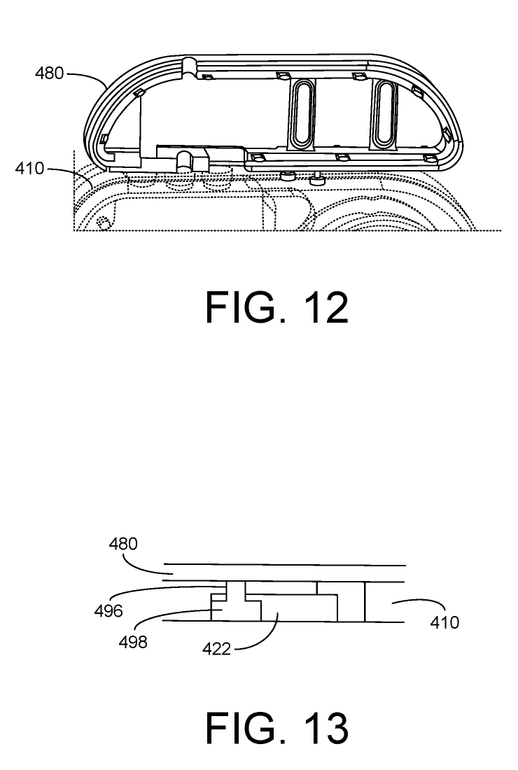

As best illustrated in FIGS. 10-12, the support member 480 may be placed such that the first projection 492 of the support member 480 aligns with the first portion 426 of the first slot 422 of the housing 410. In the illustrated embodiment, the second projection 494 of the support member 480 aligns with the second slot 424 of the housing 410. The projections of the support member 480 may then be inserted into the slots of the housing by moving the support member 480 towards the housing 410. The support member 480 may then be slid or otherwise moved with respect to the housing 410 in the direction of arrow A. This movement will move the portion of the projections of the support member 480 that extends from the slots towards the second portion of the slots. Accordingly, the support member 480 is coupled to the housing 410. In some implementations, the projections of the support member 480 are frictionally coupled within the slots to couple the support member 480 to the housing 410.

FIG. 13 is a cross-sectional view that illustrates the projections of the support member 480 within the slots of the housing 410 once the support member 480 has been moved with respect to the housing 410. As illustrated, the head portion 498 of the first projection 492 is entirely disposed within the first slot 422 and the stem portion 496 of the first projection 492 extends through an opening of the first slot 422.

In some implementations, once the charging antenna is coupled to the support member 480 and the support member 480 is coupled to the housing 410, the header portion may be formed around the charging antenna, the communication antenna, and the support member 480. For example, a material, such as silicone, may be placed or disposed around the charging antenna, the communication antenna, and the support member 480 to form the header portion.

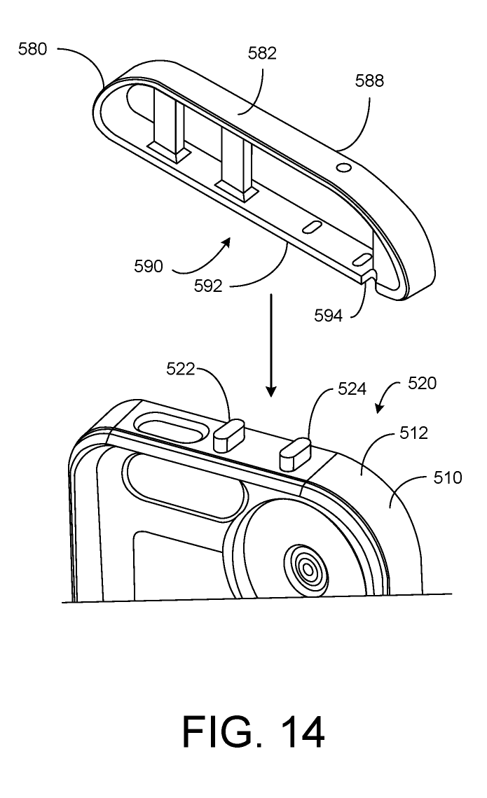

FIG. 14 is a perspective view of a support member 580 and a housing 510. As described for the above implementations, the support member 580 is disposed within a header portion and is configured to engage a charging antenna. In some implementations, the support member 580 is configured to engage the charging antenna to provide support to the charging antenna during the manufacturing process. For example, the support member 580 may provide support to the antenna while the silicone or other transparent material is disposed around the charging antenna. Additionally, in some implementations, the support member 580 is configured to keep or help keep a first portion of the charging antenna spaced from a second portion of the charging antenna.

The support member 580 defines a first groove 582 and a second groove (not illustrated in FIG. 14). The first groove 582 is disposed on a first side portion 588 of the support member 580. The second groove is disposed on a second side portion (opposite the first side portion) of the support member 580.

In some implementation, a first portion of the charging antenna is disposed within the first groove 582. A second portion of the charging antenna is disposed within the second groove. The first portion of the charging antenna may thus be disposed apart from or spaced from the second portion of the charging antenna. Specifically, a portion of the support member 580 is disposed between the first portion of the charging antenna and the second portion of the charging antenna.

In some implementations, the first portion of the charging antenna is configured to be coupled within the first groove 582. For example, in some implementations, the first portion of the charging antenna is configured to be frictionally coupled within the first groove 582. In other implementations, another coupling mechanism, such as an adhesive or other material, may be used to help retain the first portion of the charging antenna within the first groove 582.

In the illustrated implementation, the support member 580 is coupled to a top surface or portion 512 of the housing 510. Specifically, the support member 580 includes an engagement portion 590 that is configured to engage an engagement portion 520 of the housing 510. For example, the engagement portion 590 of the support member 580 may engage the engagement portion bof the support member 580 to couple the support member 580 to the housing 510.

The engagement portion 590 of the support member 580 includes a first opening 592 and a second opening 594. The engagement portion 520 of the housing 510 includes a first projection 522 and a second projection 524. The first opening 592 of the support member 580 is configured to receive the first projection 522 of the housing 510 to help couple the support member 580 to the housing 510. Similarly, the second opening 594 of the support member 580 is configured to receive the second projection 524 of the housing 510 to help couple the support member 580 to the housing 510. In some implementations, the projections are frictionally coupled within the openings. In other implementations, another method, such as glue or another adhesive, is used to couple the projections within the openings.

In some implementations, once the charging antenna is coupled to the support member 580 and the support member 580 is coupled to the housing 510, the header portion may be formed around the charging antenna, the communication antenna, and the support member 580. For example, a material, such as silicone, may be placed or disposed around the charging antenna, the communication antenna, and the support member 580 to form the header portion.

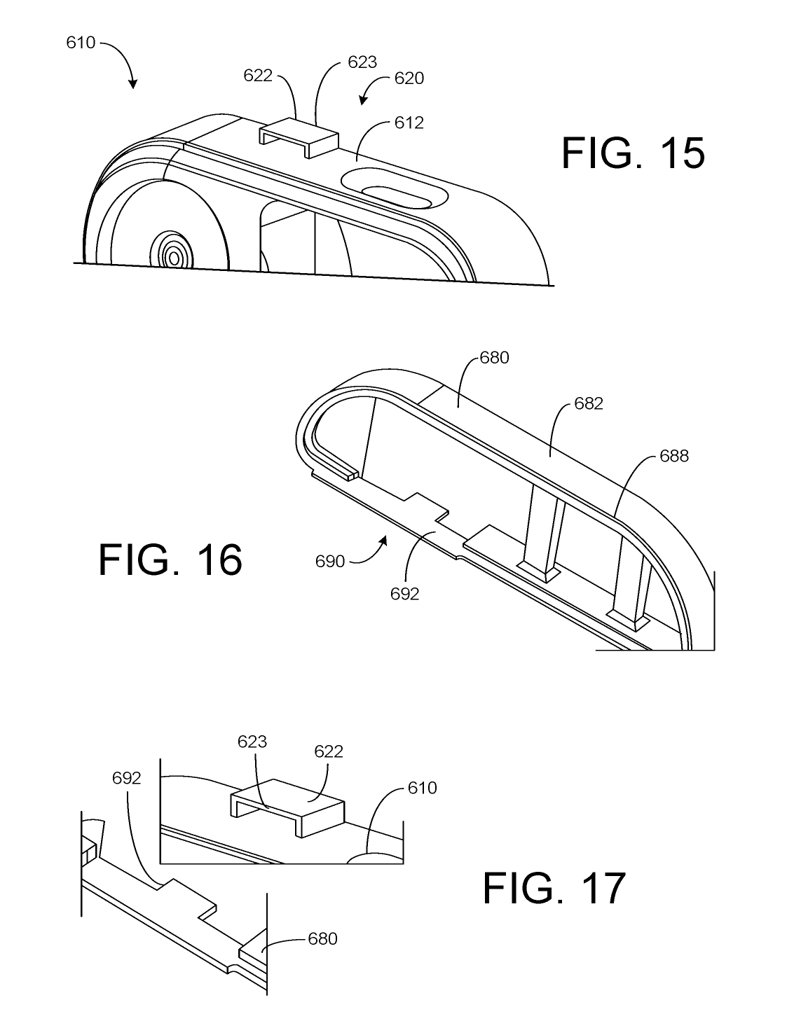

FIGS. 15-17 illustrate a support member 680 and a housing 610. As described for the above implementations, the support member 680 is disposed within a header portion and is configured to engage a charging antenna. In some implementations, the support member 680 is configured to engage the charging antenna to provide support to the charging antenna during the manufacturing process. For example, the support member 680 may provide support to the antenna while the silicone or other transparent material is disposed around the charging antenna. Additionally, in some implementations, the support member 680 is configured to keep or help keep a first portion of the charging antenna spaced from a second portion of the charging antenna.

The support member 680 defines a first groove 682 and a second groove (not illustrated). The first groove 682 is disposed on a first side portion 688 of the support member 680. The second groove is disposed on a second side portion (opposite the first side portion) of the support member 680.

In some implementation, a first portion of the charging antenna is disposed within the first groove 682. A second portion of the charging antenna is disposed within the second groove. The first portion of the charging antenna may thus be disposed apart from or spaced from the second portion of the charging antenna. Specifically, a portion of the support member 680 is disposed between the first portion of the charging antenna and the second portion of the charging antenna.

In some implementations, the first portion of the charging antenna is configured to be coupled within the first groove 682. For example, in some implementations, the first portion of the charging antenna is configured to be frictionally coupled within the first groove 682. In other implementations, another coupling mechanism, such as an adhesive or other material, may be used to help retain the first portion of the charging antenna within the first groove 682.

In the illustrated implementation, the support member 680 is coupled to a top surface or portion 612 of the housing 610. Specifically, the support member 680 includes an engagement portion 690 that is configured to engage an engagement portion 620 of the housing 610. For example, the engagement portion 690 of the support member 680 may engage the engagement portion 620 of the support member 680 to couple the support member 680 to the housing 610.

The engagement portion 690 of the support member 680 includes a projection 692. The engagement portion 620 of the housing 610 includes a projection 622 that defines a receiving portion 623. The receiving portion 623 of the projection 622 of the housing 610 is configured to receive the projection 622 of the support member 680 to help couple the support member 680 to the housing 610.

In some implementations, once the charging antenna is coupled to the support member 680 and the support member 680 is coupled to the housing 610, the header portion may be formed around the charging antenna, the communication antenna, and the support member 680. For example, a material, such as silicone, may be placed or disposed around the charging antenna, the communication antenna, and the support member 680 to form the header portion.

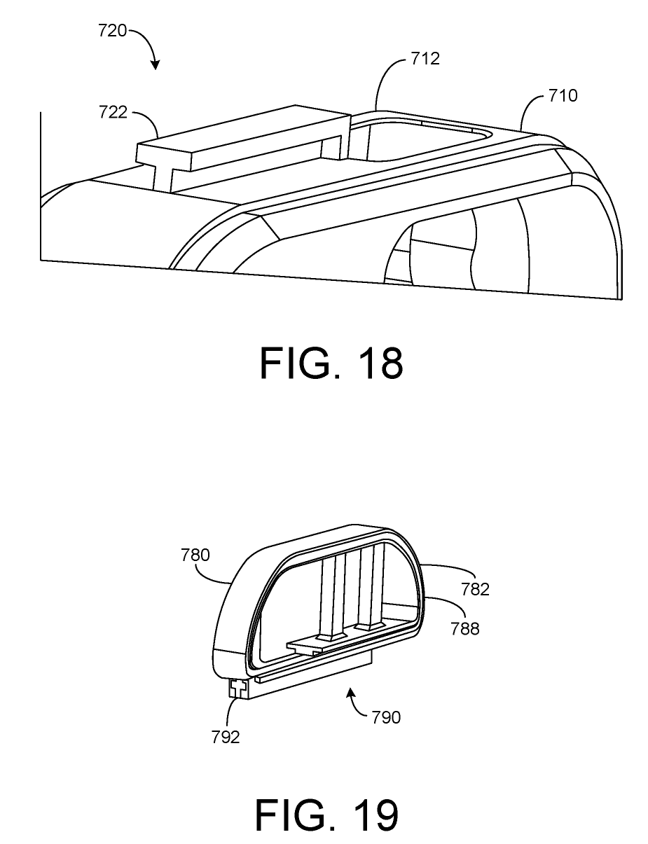

FIGS. 18 and 19 illustrate a support member 780 and a housing 710. As described for the above implementations, the support member 780 is disposed within a header portion and is configured to engage a charging antenna. In some implementations, the support member 780 is configured to engage the charging antenna to provide support to the charging antenna during the manufacturing process. For example, the support member 780 may provide support to the antenna while the silicone or other transparent material is disposed around the charging antenna. Additionally, in some implementations, the support member 780 is configured to keep or help keep a first portion of the charging antenna spaced from a second portion of the charging antenna.

The support member 780 defines a first groove 782 and a second groove (not illustrated). The first groove 782 is disposed on a first side portion 788 of the support member 780. The second groove is disposed on a second side portion (opposite the first side portion) of the support member 780.

In some implementation, a first portion of the charging antenna is disposed within the first groove 782. A second portion of the charging antenna is disposed within the second groove. The first portion of the charging antenna may thus be disposed apart from or spaced from the second portion of the charging antenna. Specifically, a portion of the support member 780 is disposed between the first portion of the charging antenna and the second portion of the charging antenna.

In some implementations, the first portion of the charging antenna is configured to be coupled within the first groove 782. For example, in some implementations, the first portion of the charging antenna is configured to be frictionally coupled within the first groove 782. In other implementations, another coupling mechanism, such as an adhesive or other material, may be used to help retain the first portion of the charging antenna within the first groove 782.

In the illustrated implementation, the support member 780 is coupled to a top surface or portion 712 of the housing 710. Specifically, the support member 780 includes an engagement portion 790 that is configured to engage an engagement portion 720 of the housing 710. For example, the engagement portion 790 of the support member 780 may engage the engagement portion 720 of the support member 780 to couple the support member 780 to the housing 710.

The engagement portion 790 of the support member 780 includes a receiving portion 792. The engagement portion 720 of the housing 710 includes a projection 722. The receiving portion 792 of the support member 780 is configured to receive the projection 722 of the housing 710 to help couple the support member 780 to the housing 710. In the illustrated implementation, the receiving portion 792 is configured to slidingly receive the projection 722.

In some implementations, once the charging antenna is coupled to the support member 780 and the support member 780 is coupled to the housing 710, the header portion may be formed around the charging antenna, the communication antenna, and the support member 780. For example, a material, such as silicone, may be placed or disposed around the charging antenna, the communication antenna, and the support member 780 to form the header portion.

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. A bodily implant, comprising:
a housing defining a cavity, the housing having an engagement portion;
an electronic control system at least partially disposed within the cavity;
a recharge antenna; and
a support member having an engagement portion, the engagement portion of the support member configured to engage the engagement portion of the housing to couple the support member to the housing.

2. The bodily implant of claim 1, wherein the support member defines a groove, the groove being configured to receive at least a portion of the recharge antenna.

3. The bodily implant of claim 1, further comprising:
a fluid control system, at least a portion of the fluid control system being disposed within the cavity defined by the housing.

4. The bodily implant of claim 1, further comprising:
an inflatable member configured to be placed in an inflated configuration and a deflated configuration.

5. The bodily implant of claim 1, further comprising:
a fluid reservoir and an inflatable member.

6. The bodily implant of claim 1, further comprising:
a fluid reservoir operatively coupled to the housing, and the bodily implant includes an inflatable member operatively coupled to the housing.

7. The bodily implant of claim 1, wherein the bodily implant is a penile implant.

8. The bodily implant of claim 1, wherein the bodily implant is an artificial sphincter.

9. The bodily implant of claim 1, wherein the coupling portion of the support member includes a projection, and the projection has a stem portion and a head portion.

10. The bodily implant of claim 1, wherein the coupling portion of the support member includes a projection, the projection has a stem portion and a head portion, the stem portion of the projection has a first size, the head portion of the projection has a second size, the second size being larger than the first size.

11. The bodily implant of claim 1, wherein the coupling portion of the housing includes a slot, the slot is configured to receive the coupling portion of the support member.

12. The bodily implant of claim 1, wherein the coupling portion of the housing includes a slot, the slot includes a first portion having a first size and a second portion having a second size, the first size being larger than the second size.

13. The bodily implant of claim 1, wherein the coupling portion of the housing is configured to receive a portion of the coupling portion of the support member to couple the support member to the housing.

14. The bodily implant of claim 1, wherein the coupling portion of the support member is configured to receive a portion of the coupling portion of the housing to couple the support member to the housing.

15. The bodily implant of claim 1, wherein the coupling portion of the housing includes a projection, the coupling portion of the support member includes an opening, the opening being configured to receive a portion of the projection of the coupling portion of the housing.

16. A bodily implant, comprising:
a housing defining a cavity, the housing defining a slot;
an electronic control system at least partially disposed within the cavity, the electronic control system includes a power storage device;
a recharge antenna; and
a support member having a projection, the slot being configured to receive at least a portion of the projection to couple the support member to the housing.

17. The bodily implant of claim 16, wherein the projection of the support member has a stem portion and a head portion, the stem portion of the projection has a first size, the head portion of the projection has a second size, the second size being larger than the first size.

18. The bodily implant of claim 16, wherein the slot includes a first portion having a first size and a second portion having a second size, the first size being larger than the second size.

19. The bodily implant of claim 16, wherein the support member defines a groove, the groove being configured to receive at least a portion of the recharge antenna.

20. The bodily implant of claim 16, further comprising:
a fluid reservoir and an inflatable member.
